SEQUENCE LISTING (84 sequences)

SEQ ID NO: 1 - protein, 7 aa, Homo sapiens
KMYFNLI

SEQ ID NO: 2 - protein, 12 aa, Homo sapiens
SYFVGKMYFNLI

SEQ ID NO: 3 - protein, 7 aa, Homo sapiens
TKFLSYK

SEQ ID NO: 4 - protein, 13 aa, Homo sapiens
LLLALAGLLAILA

SEQ ID NO: 5 - protein, 5 aa, Homo sapiens
LLSYF

SEQ ID NO: 6 - protein, 8 aa, Homo sapiens
SLMFMQWG

SEQ ID NO: 7 - protein, 5 aa, Homo sapiens
FVTGV

SEQ ID NO: 8 - protein, 5 aa, Homo sapiens
LTSFV

SEQ ID NO: 9 - protein, 6 aa, Homo sapiens
LGLLAV

SEQ ID NO: 10 - protein, 11 aa, Homo sapiens
IVGAMVQIITY

SEQ ID NO: 11 - protein, 6 aa, Homo sapiens
VFTNAF

SEQ ID NO: 12 - protein, 14 aa, Homo sapiens
VFFASWRVVLEGGI

SEQ ID NO: 13 - protein, 18 aa, Homo sapiens
LLWMGLVLALALALALAL

SEQ ID NO: 14 - protein, 5 aa, Homo sapiens
LFTAI

SEQ ID NO: 15 - protein, 5 aa, Homo sapiens
ILFLT

SEQ ID NO: 16 - protein, 9 aa, Homo sapiens
ALTTVTALV

SEQ ID NO: 17 - protein, 6 aa, Homo sapiens
NFWLLI

SEQ ID NO: 18 - protein, 6 aa, Homo sapiens
LAVAFL

SEQ ID NO: 19 - protein, 9 aa, Homo sapiens
LFQTFWFLY

SEQ ID NO: 20 - protein, 5 aa, Homo sapiens
YVAVA

SEQ ID NO: 21 - protein, 6 aa, Homo sapiens
VTAMLL

SEQ ID NO: 22 - protein, 11 aa, Homo sapiens
TTAVALAYGIY

SEQ ID NO: 23 - protein, 5 aa, Homo sapiens
NVVFV

SEQ ID NO: 24 - protein, 6 aa, Homo sapiens
VLATAF

SEQ ID NO: 25 - protein, 6 aa, Homo sapiens
MLNLYI

SEQ ID NO: 26 - protein, 12 aa, Homo sapiens
VLFCLCGCLAVV

SEQ ID NO: 27 - protein, 6 aa, Homo sapiens
VLMAAA

SEQ ID NO: 28 - protein, 5 aa, Homo sapiens
YFWGL

SEQ ID NO: 29 - protein, 5 aa, Homo sapiens
LVVFS

SEQ ID NO: 30 - protein, 7 aa, Homo sapiens
LYSAIGI

SEQ ID NO: 31 - protein, 6 aa, Homo sapiens
FIVAAV

SEQ ID NO: 32 - protein, 5 aa, Homo sapiens
ATYYF

SEQ ID NO: 33 - protein, 5 aa, Homo sapiens
SWLYL

SEQ ID NO: 34 - protein, 5 aa, Homo sapiens
SWLYL

SEQ ID NO: 35 - protein, 5 aa, Homo sapiens
LSLIL

SEQ ID NO: 36 - protein, 5 aa, Homo sapiens
IAVIG

SEQ ID NO: 37 - protein, 5 aa, Homo sapiens
LFFIN

SEQ ID NO: 38 - protein, 5 aa, Homo sapiens
FTFAT

SEQ ID NO: 39 - protein, 5 aa, Homo sapiens
AAICF

SEQ ID NO: 40 - protein, 8 aa, Homo sapiens
AALSYGFY

SEQ ID NO: 41 - protein, 6 aa, Homo sapiens
VAFCLK

SEQ ID NO: 42 - protein, 7 aa, Homo sapiens
VQFGVMI

SEQ ID NO: 43 - protein, 6 aa, Homo sapiens
YLFSVS

SEQ ID NO: 44 - protein, 8 aa, Homo sapiens
AILSFVGY

SEQ ID NO: 45 - protein, 5 aa, Homo sapiens
VLCLM

SEQ ID NO: 46 - protein, 5 aa, Homo sapiens
FLNVY

SEQ ID NO: 47 - protein, 12 aa, Homo sapiens
LFGMLGFLGVAL

SEQ ID NO: 48 - protein, 8 aa, Homo sapiens
LGYLSFLA

SEQ ID NO: 49 - protein, 10 aa, Homo sapiens
IVGVAFFNVL

SEQ ID NO: 50 - protein, 8 aa, Homo sapiens
ACVAWYWW

SEQ ID NO: 51 - protein, 6 aa, Homo sapiens
FTVVVL

SEQ ID NO: 52 - protein, 5 aa, Homo sapiens
ATFTV

SEQ ID NO: 53 - protein, 5 aa, Homo sapiens
VVAIL

SEQ ID NO: 54 - protein, 7 aa, Homo sapiens
WYMSTLY

SEQ ID NO: 55 - protein, 6 aa, Homo sapiens
IFGMLI

SEQ ID NO: 56 - protein, 5 aa, Homo sapiens
LFTCL

SEQ ID NO: 57 - protein, 6 aa, Homo sapiens
FFMGTV

SEQ ID NO: 58 - protein, 14 aa, Homo sapiens
FLMGVWGSAFSILF

SEQ ID NO: 59 - protein, 6 aa, Homo sapiens
MIMALV

SEQ ID NO: 60 - protein, 5 aa, Homo sapiens
LIISF

SEQ ID NO: 61 - protein, 10 aa, Homo sapiens
TIIHFVLANI

SEQ ID NO: 62 - protein, 8 aa, Homo sapiens
FAWGVVTC

SEQ ID NO: 63 - protein, 5 aa, Homo sapiens
TVGVC

SEQ ID NO: 64 - protein, 5 aa, Homo sapiens
YSWQC

SEQ ID NO: 65 - protein, 5 aa, Homo sapiens
YLFYP

SEQ ID NO: 66 - protein, 12 aa, Homo sapiens
LTLLLLLMAAVV

SEQ ID NO: 67 - protein, 5 aa, Homo sapiens
FQYCL

SEQ ID NO: 68 - protein, 9 aa, Homo sapiens
AALSYGFYG

SEQ ID NO: 69 - protein, 134 aa, Apis mellifera
IIYPGTLWCGHGNKSSGPNELGRFKHTDACCRTHDMCPDVMSAGESKHGLTNTASHTRLSCDCDDKFYDCLKNSADTISSYFVGKMYFNLIDTKCYKLEHPVTGCGERTEGRCLHYTVDKSKPKVYQWFDLRKY

SEQ ID NO: 70 - protein, 147 aa, Homo sapiens
MKTLLLLAVIMIFGLLQLLQAHGNLVNFHRMIKLTTGKEAALSYGFYGCHCGVGGRGSPKDATDRCCVTHDCCYKRLEKRGCGTKFLSYKFSNSGSRITCAKQDSCRSQLCECDKAAATCFARNKTTYNKKYQYYSNKHCRGSTPRS

SEQ ID NO: 71 - protein, 148 aa, Homo sapiens
MKLLVLAVLLTVAAADSGISPRAVWQFRKMIKCVIPGSDPFLEYNNYGCYCGLGGSGTPVDELDKCCQTHDNCYDQAKKLDSCKFLLDNPYTHTYSYSCSGSAITCSSKNKECEAFICNCDRNAAICFSKAPYNKAHKNLDTKKYCQS

SEQ ID NO: 72 - protein, 140 aa, Homo sapiens
MKTLLLLAVIMIFGLLQAHGNLVNFHRMLKLTTGKEAALSYGFYGCHCGVGGRGSPKDATDRCCVTHDCCYKRLEKRGCGTKFLSYKFSNSGSRLTCAKQDSCRSQLCECDKAAATCFARNKTTYQYYSNKHCRGSTPRC

SEQ ID NO: 73 - protein, 145 aa, Homo sapiens
MELALLCGLVVMAGVIPIQGGILNLNKMVKQVTGKMPILSYWPYGCHCGLGGRGQPKDATDWCCQTHDCCYDHLKTQGCSIYKDYYRYNFSQGNIHCSDKGSWCEQQLCACDKEVAFCLKRNLDTYQKRLRFYWRPHCRGQTPGC

SEQ ID NO: 74 - protein, 141 aa, Homo sapiens
MKSPHVLVFLCLLVALVTGNLVQFGVMIEKMTGKSALQYNDYGCYCGIGGSHWPVDQTDWCCHAHDCCYGRLEKLGCEPKLEKYLFSVSERGWCAGRTTCQRLTCECDKRAALCFRRNLGTYNRKYAHYPNKLCTGPTPPC

SEQ ID NO: 75 - protein, 211 aa, Homo sapiens
MADGAKANPKGFKKKVLDRCFSGWRGPRFGASCPSRTSRSSLGMKKFFTVAILAGSVLSTAHGSLLNLKAMVEAVTGRSAILSFVGYGCYCGLGGRGQPKDEVDWCCHAHDCCYQELFDQGCHPYVDHYDHTIENNTELVCSDLNKTECDKQTCMCDKNMVLCLMNQTYREEYRGFLNVYCQGPTPNCSIYEPPPEEVTCSHQSPAPPAPP

SEQ ID NO: 76 - protein, 509 aa, Homo sapiens
MGVQAGLFGMLGFLGVALGGSPALRWYRTSCHLTKAVPGNPLGYLSFLAKDAQGLALIHARWDAHRRLQSCSWEDEPELTAAYGALCAHETAWGSFIHTPGPELQRALATLQSQWEACRALEESPAGARKKRAAGQSGVPGGGHQREKRGWTMPGTLWCGVGDSAGNSSELGVFQGPDLCCREHDRCPQNISPLQYNYGIRNYRFHTISHCDCDTRFQQCLQNQHDSISDIVGVAFFNVLEIPCFVLEEQEACVAWYWWGGCRMYGTVPLARLQPRTFYNASWSSRATSPTPSSRSPAPPKPRQKQHLRKGPPHQKGSKRPSKANTTALQDPMVSPRLDVAPTGLQGPQGGLKPQGARWVCRSFRRHLDQCEHQIGPREIEFQLLNSAQEPLFHCNCTRRLARFLRLHSPPEVTNMLWELLGTTCFKLAPPLDCVEGKNCSRDPRAIRVSARHLRRLQQRRHQLQDKGTDERQPWPSEPLRGPMSFYNQCLQLTQAARRPDRQQKSWSQ

SEQ ID NO: 77 - protein, 748 aa, Homo sapiens
MSFIDPYQHIIVEHQYSHKFTVVVLRATKVTKGAFGDMLDTPDPYVELFISTTPDSRKRTRHFNNDINPVWNETFEFILDPNQENVLEITLMDANYVMDETLGTATFTVSSMKVGEKKEVPFIFNQVTEMVLEMSLEVCSCPDLRFSMALCDQEKTFRQQRKEHIRESMKKLLGPKNSEGLHSARDVPVVAILGSGGGFRAMVGFSGVMKALYESGILDCATYVAGLSGSTWYMSTLYSHPDFPEKGPEEINEELMKNVSHNPLLLLTPQKVKRYVESLWKKKSSGQPVTFTDIFGMLIGETLIHNRMNTTLSSLKEKVNTAQCPLPLFTCLHVKPDVSELMFADWVEFSPYEIGMAKYGTFMAPDLFGSKFFMGTVVKKYEENPLHFLMGVWGSAFSILFNRVLGVSGSQSRGSTMEEELENITTKHIVSNDSSDSDDESHEPKGTENEDAGSDYQSDNQASWIHRMIMALVSDSALFNTREGRAGKVHNFMLGLNLNTSYPLSPLSDFATQDSFDDDELDAAVADPDEFERLYEPLDVKSKKIHVVDSGLTFNLPYPLIRPQRGVDLIISFDFSARPSDSSPPFKELLLAEKWAKMNKLPFPKIDPYVFDREGLKECYVFKPKNPDMEKDCPTIIHFVLANINFRKYRAPGVPRETEEEKEIADFDIFDDPESPFSTFNFQYPNQAFKRLHDLMHFNTLNNIDVLKEAMVESIEYRRQNPSRCSVSLSNVEARRFFNKEFLSKPKA

SEQ ID NO: 78 - protein, 138 aa, Homo sapiens
MKGLLPLAWFLACSVPAVQGGLLDLKSMIEKVTGKNALTNYGFYGCYCGWGGRGTPKDGTDWCCWAHDHCYGRLEEKGCNIRTQSYKYRFAWGVVTCEPGPFCHVNLCACDRKLVYCLKRNLRSYNPQYQYFPNILCS

SEQ ID NO: 79 - protein, 165 aa, Homo sapiens
MGPLPVCLPIMLLLLLPSLLLLLLLPGPGSGEASRILRVHRRGILELAGTVGCVGPRTPIAYMKYGCFCGLGGHGQPRDAIDWCCHGHDCCYTRAEEAGCSPKTERYSWQCVNQSVLCGPAENKCQELLCKCDQEIANCLAQTEYNLKYLFYPQFLCEPDSPKCD

SEQ ID NO: 80 - protein, 189 aa, Homo sapiens
MALLSRPALTLLLLLMAAVVRCQEQAQTTDWRATLKTIRNGVHKIDTYLNAALDLLGGEDGLCQYKCSDGSKPFPRYGYKPSPPNGCGSPLFGVHLNIGIPSLTKCCNQHDRCYETCGKSKNDCDEEFQYCLSKICRDVQKTLGLTQHVQACETTVELLFDSVIHLGCKPYLDSQRAACRCHYEEKTDL

SEQ ID NO: 81 - protein, 440 aa, Homo sapiens
MVPPKLHVLFCLCGCLAVVYFFDWQYLNPVAHMKSSAWVNKIQVLMAAASFGQTKIPRGNGPYSVGCTDLMFDHTNKGTFLRLYYPSQDNDRLDTLWIPNKEYFWGLSKFLGTHWLMGNILRLLFGSMTTPANWNSPLRPGEKYPLVVFSHGLGAFRTLYSAGIDLASHGFIVAAVEHRDRSASATYYFKDQSAAEIGDKSWLYLRTLKQEEETHIRNEQVRQRAKECSQALSLILDIDHGKPVKNALDLKFDMEQLKDSIDREKIAVIGHSFGGATVIQTLSEDQRFRCGIALDAWMFPLGDEVYSRIPQPLFFINSEYFQYFANIIKMKKCYSPDKERKMITIRGSVHQNFADFTFATGKIIGHMLKLKGDIDSNVAIDLSNKASLAFLQKHLGLHKDFDQWDCLIEGDDENLIPGTNINTTNQHIMLQNSSGIEKYN

SEQ ID NO: 82 - protein, 744 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 83 - protein, 629 aa, Homo sapiens
MPRYGASLRQSCPRSGREQGQDGTAGAPGLLWMGLVLALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESIPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSRSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 84 - protein, 701 aa, Homo sapiens [sequence omitted]

(19) United States
(12) Patent Application Publication
(10) Pub. No.: US 2013/0079493 A1
(43) Pub. Date: Mar. 28, 2013

(54) Method for prepairing peptide inhibitors of a lipid-activated enzyme and peptides produced by same

(71) Applicant: Paavo Kinnunen, Espoo (FI)

(72) Inventor: Paavo Kinnunen, Espoo (FI)

(73) Assignee: ESTAJA OY, Helsinki (FI)

(21) Appl. No.: 13/701,719

(22) PCT Filed: Jun. 3, 2011
(86) PCT No.: PCT/FI11/50519

§ 371 Date: Dec. 14, 2012

(30) Foreign Application Priority Data
Jun. 3, 2010 (FI) 20105629

(51) Int. Cl. C07K 7/06; C07K 7/08; C07K 19/00; G06F 19/16

(52) U.S. Cl. CPC C07K 7/06; G06F 19/16; C07K 7/08; C07K 19/00
USPC 530/326; 530/333; 530/329; 530/328; 530/327; 530/330; 530/300

(57) ABSTRACT

The present invention is based on the discovery of a mechanism mediating the formation of amyloid-type aggregates of lipid-activated enzymes. The invention discloses a method for preparing inhibitors of said enzymes and provides peptide inhibitors having potential for therapeutic use. The method comprises the identification of aggregation-prone regions in the amino acid sequence of the enzyme by the use of a suitable computer algorithm and designing a peptide based on the found aggregation-prone region.

### FIELD OF THE INVENTION

This invention relates to the field of enzymology. In particular, the present invention is based on the discovery of mechanisms mediating the formation of amyloid-type aggregates of lipid-activated enzymes. The invention discloses a method for preparing inhibitors of said enzymes and provides peptide inhibitors having potential for therapeutical use.

### BACKGROUND OF THE INVENTION

Shimizu, 2009, discloses that prostaglandins, leukotrienes, platelet-activating factor, lysophosphatidic acid, sphingosine 1-phophate, and endocannabinoids, collectively referred to as lipid mediators, play pivotal roles in human immune regulation and self-defense. These lipid mediators are produced by multistep enzymatic pathways involving lipid-activated enzymes such as phospholipases. The author summarizes that researchers need to develop specific inhibitors and receptor agonists and antagonists of the lipid-activated enzymes since these would have great potential as a therapeutic approach to disease.

In Code et al., 2008, it is disclosed that activity of phospholipase A2 (PLA2) is dependent on the process of amyloid formation of the enzyme explaining its lag-burst behaviour in enzymatic catalysis. The following route for the activation of PLA2 was discussed: 1) the soluble monomeric enzyme rapidly binds to the substrate; 2) after binding a slow dimerization of the enzyme takes place, at this stage the enzyme shows low catalytic activity; 3) formation of “molten dimers” before the burst of activity; 4) formation of protofibrillar oligomers of PLA2 with high catalytic activity; and finally 5) emergence of amyloid-like fibrils devoid of enzymatic activity.

Several amyloidogenic peptides are known to enhance the activity of PLA2, such as temporin B (temB) and temporin L. It has been hypothesized that the formation of heterooligomers by PLA2 and temB would be responsible for the activation by temB of PLA2, promoting enzyme aggregation into an active conformation (Code et al., 2009).

The present invention is directed to a method for producing peptide inhibitors of PLA2 and other lipid-activated enzymes by identifying aggregation-prone regions of these enzymes responsible for the formation of inactive amyloids and designing a peptide inhibitor accordingly. The present invention is able to show that this approach provides effective inhibitors of enzyme activity.

### BRIEF DESCRIPTION OF THE DRAWINGS

FIG. 1. Activity of phospholipase A₂ of bee venom in the presence of peptide inhibitor having sequence KMYFNLI (SEQ ID NO:1).

FIG. 2. Activity of phospholipase A₂ (of human tears) in the presence of peptide inhibitor having sequence AALSYGFYG (SEQ ID NO:68).

### DETAILED DESCRIPTION OF THE INVENTION

The present invention discloses a method for preparing peptides capable of efficiently inhibiting the catalytic activity of lipid-activated enzymes and provides peptides made by said method. The expression “lipid-activated enzyme” refers herein to enzymes that specifically recognize a structure or bond of a lipid and require this interaction for maximal activity. Examples of such lipids are fatty acyls, glycerolipids, glycerophospholipids, sphingolipids, saccharolipids and polyketides. Examples of lipid-activated enzymes are carboxylic ester hydrolases (EC 3.1.1), such as phospholipases A1, A2, B and PAF acetylhydrolase, as well as heat shock protein 70 and sphingomyelins. Particularly, some of the lipid-activated enzymes as defined in the present invention are enzymes which generate lipid mediators, such as fatty acids, phospholipids and lysophospholipids (see Shimizu, 2009)

The mechanism of activation of phospholipase A2 as described by Code et al. (2008) requires direct protein-protein interactions. These interactions also need to involve protein sequences capable of causing aggregation and amyloid type oligomerization of the enzyme. When this type of sequences were sought by the use of a computer algorithm in the structure of bee venom phospholipase A2, a stretch of residues 78-91 fulfilling these criteria was found.

A suitable computer algorithm for use in the present method is preferably selected from the group consisting of: AGGRESCAN (Conchillo-Sole et al., 2007), PASTA (Trovato et al., 2007) and TANGO (Rousseau et al., 2006; Fernandez-Escamilla et al., 2004; Linding et al., 2004). These computer algorithms are designed and generally utilized as web-based software for the prediction of aggregation-prone segments in protein sequences.

The peptide inhibitor of the invention is prepared based on the found aggregation-prone segment so that the peptide sequence is identical to the amino acid sequence of the segment. The length of the peptide may vary: the peptide can be as long as the aggregation-prone segment, or it can correspond only to a part of the segment. Preferably, the peptides are 5-12 amino acids long. Peptides of the invention can be synthesized by well-known methods (see, e.g., Atherton and Sheppard, 1989).

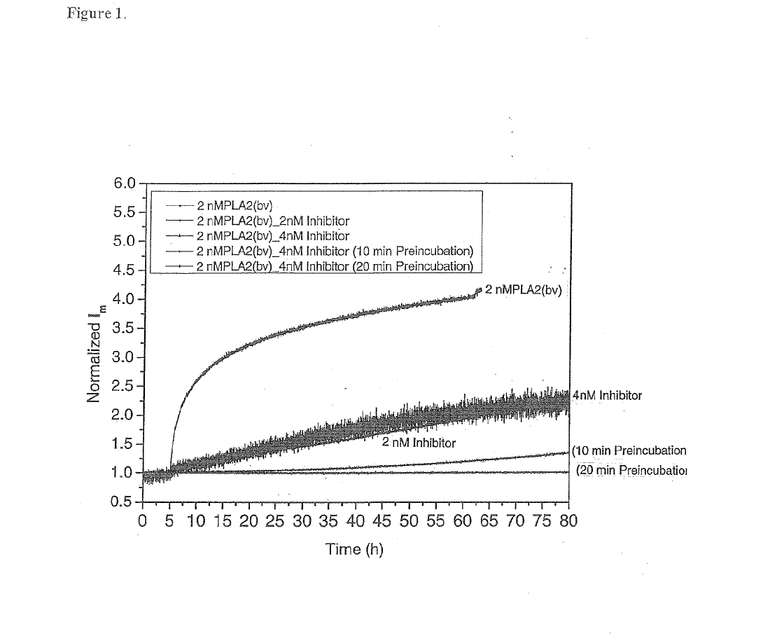

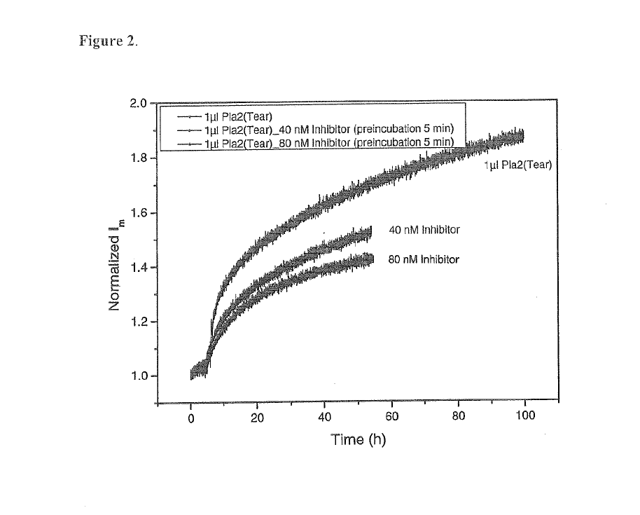

Further along the lines of the present invention, synthetic peptides of this structure are readily expected to inhibit proper lipid-activated enzyme, such as PLA2-PLA2, contacts. The latter was verified using a short synthetic peptide corresponding to residues 85-91 (KMYFNLI, SEQ ID NO:1) of the bee venom PLA2 enzyme, which upon preincubation with the enzyme protein was capable of causing complete inhibition (see FIG. 1). Notably, the inhibition was observed at equimolar concentrations with the enzyme (2 nanomolar), making this the most potent inhibitor described so far. The same experiment was subsequently performed for the human secretory PLA2 present in tear fluid. For this enzyme, an amyloid aggregation causing region of residues 17-25 was found (AALSYGFYG, SEQ ID NO:68) and the synthetic corresponding peptide also inhibited the tear fluid PLA2 activity (FIG. 2).

This particular mechanism of enzyme activity control can be expected to be very widely found in nature. Accordingly, identification of this type of sequences in lipid-activated enzymes can be used to obtain very specific and powerful synthetic peptide inhibitors.

Furthermore, the peptides can be made with additional cell membrane permeating sequences, so that the inhibitors can enter cells, i.e. with transport peptides, see, e.g., U.S. Pat. No. 7,265,092. An example of a transporter peptide is a peptide which facilitates cellular uptake of an inhibitor peptide which is chemically associated or bonded to the transporter peptide.

Along these lines, we identified the following amyloid aggregation sequences in the following human enzymes: myeloperoxidase, acid sphingomyelinase, and heat shock protein 70 (see Table 1 below).

Heat shock protein 70 is of particular importance as its inhibition makes cell extremely sensitive to an increase in temperature. This feature could be exploited in for instance MRI-guided HIFU therapy to more efficiently eradicate cancer.

A number of peptide inhibitor candidates for human phospholipases A were also identified with sequences fulfilling the above criteria (see Table 1). Also peptide inhibitor candidates for human PAF acetyl hydrolase were found (see Table 1).

Accordingly, the present invention is directed to a method for preparing peptide inhibitors of a lipid-activated enzyme, the method comprising the steps of:

a) identifying aggregation-prone regions in amino acid sequence of said enzyme by the use of a suitable computer algorithm;
b) designing a peptide based on the aggregation-prone region found in step a), wherein said peptide comprises the sequence of said region or a part thereof,
c) synthesizing the peptide designed in step b);
d) contacting the peptide obtained in step c) with said lipid-activated enzyme and measuring the activity of said enzyme, wherein said peptide is an inhibitor of said enzyme, if the activity of the enzyme is decreased in the presence of said peptide.

Preferably, said lipid-activated enzyme is selected from the group consisting of phospholipases, myeloperoxidase, acid sphingomyelinase, heat shock protein 70 and PAF acetylhydrolase. Peptide inhibitor candidates already designed based on the aggregation-prone regions found from these lipid-activated enzymes are listed in Table 1 below.

The present invention also provides peptides comprising amino acid sequence set forth in any one of SEQ ID NOS:1-68. Preferably, said peptide comprises or consists of amino acid sequence KMYFNLI (SEQ ID NO:1). In another preferred embodiment said peptide comprises or consists of amino acid sequence AALSYGFYG (SEQ ID NO:68).

The present invention further includes pharmaceutical compositions comprising a pharmaceutically effective amount of one or more of the above-described peptides as active ingredient. Pharmaceutical compositions according to the invention are suitable for topical, enteral, such as oral or rectal, and parenteral administration to mammals, including man, for the treatment of bee sting or other phospholipase related condition, including cancer, rheumatoid arthritis, multiple sclerosis, bronchial asthma, intestinal polyposis or pulmonary fibrosis or in combination with one or more pharmaceutically acceptable carriers.

The inventive compounds are useful for the manufacture of pharmaceutical compositions having an effective amount the compound in conjunction or admixture with excipients or carriers suitable for topical, enteral or parenteral application. Examples include tablets and gelatin capsules comprising the active ingredient together with (a) diluents; (b) lubricants, (c) binders (tablets); if desired, (d) disintegrants; and/or (e) absorbents, colorants, flavors and sweeteners. Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, the compositions may also contain other therapeutically valuable substances. The compositions are prepared according to conventional mixing, granulating or coating methods, respectively, and contain preferably about 1 to 50% of the active ingredient.

More generally, the present invention also relates to the use of the compounds of the invention for the manufacture of a medicament, in particular for the manufacture of a medicament for the treatment of the above-mentioned conditions and diseases.

The pharmaceutical composition contains a pharmaceutically effective amount of the present active agent along with other pharmaceutically acceptable exicipients, carriers, fillers, diluents and the like. The term therapeutically effective amount as used herein indicates an amount necessary to administer to a host to achieve a therapeutic result, especially an antidote effect.

As discussed above, the compounds of the present invention are useful for treating the above-mentioned conditions and diseases. Thus, the present invention further relates to a method of treating said conditions and diseases which comprises administering a therapeutically effective amount of a compound of the invention to a mammal, preferably a human, in need of such treatment.

The present invention also provides kits for use in treating bee stings or other phospholipase or lipid-activated enzyme related condition as mentioned above comprising an administration means and a container means containing a pharmaceutical composition of the present invention. Preferably, the container in which the composition is packaged prior to use will comprise a hermetically sealed container enclosing an amount of the lyophilized formulation or a solution containing the formulation suitable for a pharmaceutically effective dose thereof, or multiples of an effective dose. The composition is packaged in a sterile container, and the hermetically sealed container is designed to preserve sterility of the pharmaceutical formulation until use. Optionally, the container can be associated with administration means and/or instruction for use.

The publications and other materials used herein to illuminate the background of the invention, and in particular, to provide additional details with respect to its practice, are incorporated herein by reference. The present invention is further described in the following examples, which are not intended to limit the scope of the invention.

### EXAMPLES

### Example 1

The sequence of mature secretory phospholipase A₂ (Apis mellifica) of class III (NP_{—}001011614; XP_{—}391951; GI:58585172) consisting of amino acids 34-167 (SEQ ID NO:69) of the precursor was screened for aggregation-prone segments using CSSP, AGGRESCAN (Conchillo-Sole et al., 2007), PASTA (Trovato et al., 2007) and TANGO (Rousseau et al., 2006; Fernandez-Escamilla et al., 2004; Linding et al., 2004). The stretch of amino acids 78-91 was found corresponding to sequence SYFVGKMYFNLI (SEQ ID NO:2) comprising sequence KMYFNLI (SEQ ID NO:1) as shown below.

| | Region identified by | | | |
|---|---|---|---|---|
| | CSSP | TANGO | PASTA | AGGRESCAN |
| PLA₂ | a | b | c | d |
| >gi\|58585172:34-167 | 2-4, 6-10, | 77-92 | 78-91 p (−6.46) | 5-9, 78-91 |
| phospholipase A2 [Apis | 14-34, 37-52, | | | |
| mellifera] | 55-63, 78-92 | | | |
| IIYPGTLWCGHGNKSSGPNELG | | | | |
| RFKHTDACCRTHDMCPDVMSAG | | | | |
| ESKHGLTNTASHTRLSCDCDDK | | | | |
| FYDCLKNSADTISSYFVGKMYF | | | | |
| NLIDTKCYKLEHPVTGCGE | | | | |
| RTEGRCLHYTVDKSKPKVYQWF | | | | |
| DLRKY | | | | |

Mature Sequence PLA2 (bv)
¹IIRYPGTLWCGHGNKSSGPNELGRFKHTDACCRTHDMCPDVMSAGESKHGLTNTASHTRLSCDCDDKFYDCL
KNSADTISSYFVGKIVIYFNLIDTKCYKLEHPVTGCGERTEGRCLHYTVDKSKPKVYQWFDLRKY¹³⁴
Inhibitors
1)⁸⁵KMYFNLI⁹¹
2)⁷⁸SYFVGKMYFNLI⁹¹

### Example 2

Activity of secretory phospholipase A₂ (Apis mellifica) of class III towards C₂₈—O—PHPM was measured in the presence of peptide KMYFNLI (SEQ ID NO:1). Reaction mixture contained 2 nM of phospholipase A₂, 2 nM or 4 nM of the peptide and 1.25 μM C₂₈—O—PHPM (1-octacosanyl-2-(6-pyren-1-yl)hexanoyl-sn-glycero-3-phosphatidylmethanol) in 2.0 ml of 5 mM HEPES, 0.1 mM EDTA, 1 mM CaCl₂, pH 7.4 at 37° C. with stirring. The assay was performed with or without preincubation step. The enzymatic reaction was followed by measuring the pyrene monomer fluorescence intensity at 400 nm using a spectrofluorometer. Excitation wavelength was 343 nm and the excitation and emission slits were 10 nm. The results are shown in FIG. 1.

### Example 3

The sequence of human phospholipase A₂, content of tears (P14555), consisting of amino acids 1-144 (SEQ ID NO:70) was screened for aggregation-prone segments as described in Example 1. The stretches of amino acids 17-25 and 61-67 were found corresponding to sequences AALSYGFYG (SEQ ID NO:68) and TKFLSYK (SEQ ID NO:3), respectively, as shown below.

| | Region identified by | | |
|---|---|---|---|
| | TANGO | PASTA | AGGRESCAN |
| PLA₂ | b | c | d |
| >gi\|129483\|sp\|P14555.2\|PA2GA_HUMAN | .17-25 | 17-25, 50-70 | 5-12, 19-29, 61-67 |
| phospholipase A2 Group II PLA₂ Content of Tears | | | |
| MKTLLLLAVIMIFGLLQAHGNLVNFHRMIK | | | |
| LTTGKEAALSYGFYGCHCGVGGRGSPKDA | | | |
| TDRCCVTHDCCYKRLEKRGCGTKFLSIKF | | | |
| SNSGSRITCAKQDSCRSQLCECDKAAATCF | | | |
| ARNKTTYNKKYQYYSNKHCRGSTPRC | | | |

Inhibitor 1
(17-25 ) AALSYGFYG-NH2
Inhibitor 2
(61-67 ) TKFLSYK-NH2

### Example 4

Activity of human phospholipase A₂, content of tears (P14555), was measured in the presence of peptide AALSYGFYG (SEQ ID NO:68). Reaction mixture contained 2 nM of phospholipase of human tears, 40 nM or 80 nM of the peptide and 1.25 μM C₂₈—O—PHPM (1-octacosanyl-2-(6-pyren-1-yl)hexanoyl-sn-glycero-3-phosphatidylmethanol) in 2.0 ml of 5 mM HEPES, 0.1 mM EDTA, 1 mM CaCl₂, pH 7.4 at 37° C. with stirring. The assay was performed with or without preincubation step. The enzymatic reaction was followed as described in Example 2. The results are shown in FIG. 2.

### Example 5

The sequences of human phospholipase A2, PAF acetylhydrolase myeloperoxidase, acid sphingomyelinase, and heat shock protein 70 were screened for aggregation-prone segments using AGGRESCAN (Conchillo-Sole et al., 2007), PASTA (Trovato et al., 2007) and TANGO (Rousseau et al., 2006; Fernandez-Escamilla et al., 2004; Linding et al., 2004). The stretches found are listed in Table 1.

TABLE 1
The sequences examined and peptide candidates found.

Phospholipase A2, group IB (pancreas) [Homo sapiens]
Phospholipase A2, group IB (pancreas) [Homo sapiens]
>gi|76827695|gb|AAI06727.1|Phospholipase A2, group IB (pancreas) [Homo sapiens]
MKLLVLAVLLTVAAADSGISPR
²³AVWQFRKMIKCVIPGSDPFLEYNNYGCYCGLGGSGTPVDELDKCCQTHDNCYDQAKKLDSKFLLDN
PYTHTYSYSCSGSAITCSSKNKECEAFICNCDRNAAICFSKAPYNKAHKNLDTKKYCQS
¹⁰²AAICFI¹⁰⁶ (0.221)
phospholipase A2, group IIA precursor [Homo sapiens]
phospholipase A2, group IIA precursor [Homo sapiens]
>gi|239915991|ref|NP_001155201.1|phospholipase A2, membrane associated precursor [Homo
sapiens]
MKTLLLLAVIMIFGLLQAHG
²¹NLVNFHRMIKLTTGKEAALSYGFYGCHCGVGGRGSPKDATDRCCVTHDCCYKRLEKRGCG
TKFLSYKFSNSGSRITCAKQDSCRSQLCECDKAAATCFARNKTTYNKKYQYYSNKHCRGSTP
RC
³⁸AALSYGFY⁴⁵ (5)
group IID secretory phospholipase A2 precursor [Homo sapiens]
group IID secretory phospholipase A2 precursor [Homo sapiens]
>gi|6912596|ref|NP_036532.1|phospholipase A2, group IID precursor [Homo sapiens]
MELALLCGLVVMAGVIPIQG
²¹GILNLNKMVKQVTGKMPILSYWPYGCHCGLGGRGQPKDATDWCCQTHDCCYDHLKTQGC
SIYKDYYRYNFSQGNIHCSDKGSWCEQQLCACDKEVAFCLKRNLDTYQKRLRFYWRPHCRG
QTPGC
¹¹⁶VAFCLK¹²¹ (4.5)
group IIE secretory phospholipase A2 precursor [Homo sapiens]
group IIE secretory phospholipase A2 precursor [Homo sapiens]
>gi|7657461|ref|NP_055404.1|phospholipase A2, group IIE precursor [Homo sapiens]
MKSPHVLVFLCLLVALVTGN
²¹LVQFGVMIEKMTGKSALQYNDYGCYCGIGGSHWPVDQTDWCCHAHDCCYGRLEKLGCEPKLEKYLF
SVSERGIFCAGRTTCQRLTCECDKRAALCFRRNLGTYNRKYAHYPNKLCTGPTPPC
²³VQFGVMI²⁹ (39)
⁸⁵YLFSVS⁹⁰ (20)
group IIF secretory phospholipase A2 [Homo sapiens]
group IIF secretory phospholipase A2 [Homo sapiens]
>gi|145553989|ref|NP_073730.3|group IIF secretory phospholipase A2 [Homo sapiens]
MADGAKANPKGFKKKVLDRCFSGWRGPRFGASCPSRTSRSSLGMKKFFTVAILAGSVLSTAHG
⁶⁴SLLNLKAMVEAVTGRSAILSFVGYGCYCGLGGRGQPKDEVDWCCHAHDCCYQELFDQGCH
PYVDHYDHTIENNTEIVCSDLNKTECDKQTCMCDKNMVLCLMNQTYREEYRGFLNVYCQGP
TPNCSIYEPPPEEVTCSHQSPAPPAPP
¹⁷AILSEVGY²⁴ (62)
⁹⁸VLCLM¹⁰² (16)
¹¹³FLNVY¹¹⁷ (4)
group 3 secretory phospholipase A2 precursor [Homo sapiens]
group 3 secretory phospholipase A2 precursor [Homo sapiens]
>gi|142976884|ref|NP_056530.2|group 3 secretory phospholipase A2 precursor [Homo sapiens]
MGVQAGLFGMLGFLGVALGGSPALRWYRTSCHLTKAVPGNPLGYLSFLAKDAQGLALIHAR
WDAHRRLQSCSWEDEPELTAAYGALCAHETAWGSFIHTPGPELQRALATLQSQWEACRALEE
SPAGARKKRAAGQSGVPGGGHQREKRGWTMPGTLWCGVGDSAGNSSELGVFQGPDLCCRE
HDRCPQNISPLQYNYGIRNYRFHTISHCDCDTRFQQCLQNQHDSISDIVGVAFFNVLEIPCFVLE
EQEACVAWYWWGGCRMYGTVPLARLQPRTFYNASWSSRATSPTPSSRSPAPPKPRQKQHLR
KGPPHQKGSKRPSKANTTALQDPMVSPRLDVAPTGLQGPQGGLKPQGARWVCRSFRRHLDQ
CEHQIGPREIEFQLLNSAQEPLFHCNCTRRLARFLRLHSPPEVTNMLWELLGTTCFKLAPPLDC
VEGKNCSRDPRAIRVSARHLRRLQQRRHQLQDKGTDERQPWPSEPLRGPMSFYNQCLQLTQA
ARRPDRQQKSWSQ
⁷LFGMLGFLGVAL¹⁸ (70)
⁴²LGYLSFLA⁴⁹ (38)
²³¹IVGVAFFNVL²⁴⁰ (81)
²⁵²ACVAWYWW²⁵⁹ (91)
Cytosolic Group IV phospholipases A₂ (cPLA₂)
phospholipase A2, group IVA (cytosolic, calcium-dependent) [Homo sapiens]
phospholipase A2, group IVA (cytosolic, calcium-dependent) [Homo sapiens]
>gi|56203412|emb|CAI22252.1|phospholipase A2, group IVA (cytosolic, calcium-dependent)
[Homo sapiens]
MSFIDPYQHIIVEHQYSHKFTVVVLRATKVTKGAFGDMLDTPDPYVELFISTTPDSRKRTRHENNDINPVW
NETFEFILDPNQENVLEITLMDANYVMDETLGTATFTVSSMKVGEKKEVPFIFNQVTEMVLEMSLEVCSC
PDLRFSMALCDQEKTFRQQRKEHIRESMKKLLGPKNSEGLHSARDVPVVAILGSGGGFRAMVGFSGVMK
ALYESGILDCATYVAGLSGSTWYMSTLYSHPDFPEKGPEEINEELMKNVSHNPTLLLLTPQKVKRYVESLW
KKKSSGQPVTFTDIFGMLIGETLIHNRMNTTLSSLKEKVNTAQCPLPLFTCLHVKPDVSELMFADWVEFSP
YEIGMAKYGTFMAPDLFGSKFFMGTVVKKYEENPLRFLMGVWGSAFSILFNRVLGVSGSQSRGSTMEEE
LENITTKHIVSNDSSDSDDESHEPKGTENEDAGSDYQSDNQASWIHRMIMALVSDSALFNTREGRAGKVH
NFMLGLNLNTSYPLSPLSDFATQDSFDDDELDAAVADPDEFERIYEPLDVKSKKIHVVDSGLTFNLPYPLI
LRPQRGVDLIISFDFSARPSDSSPPFKELLLAEKWAKMNKLPFPKIDPYVFDREGLKECYVFKPKNPDMEK
DCPTIIHFVLANINFRKYRAPGVPRETEEEKEIADFDIFDDPESPFSTFNFQYPNQAFKRLHDLMHFNTLNNI
DVIKEAMVESIEYRRQNPSRCSVSLSNVEARRFFNKEFLSKPKA
²⁰FTYVVL²⁵ (82)
¹⁰⁵ATFTV¹⁰⁹ (6.5)
¹⁸⁹VVAIL¹⁹³ (85.5)
²³²WYNISTLY²³⁸ (17)
²⁹⁴IFGMLI²⁹⁹ (9)
³²⁸LFTCL³³² (5.4)
³⁷²FFMGTV³⁷⁷ (4.8)
³⁸⁸FLMGVWGSAFSILF⁴⁰¹ (55)
⁴⁶⁸MIMALV⁴⁷³ (79)
⁷⁰LIISF⁷⁴ (8.2)
¹³⁶TIIHFVLANI¹⁴⁵ (32)
phospholipase A2, group V precursor [Homo sapiens]
phospholipase A2, group V precursor [Homo sapiens]
>gi|4505853|ref|NP_000920.1|calcium-dependent phospholipase A2 precursor [Homo sapiens]
MKGLLPLAWFLACSVPAVQG (sig pept)
²⁰GLLDLKSMIEKVTGKNALTNYGFYGCYCGWGGRGTPKDGTDWCCWAHDHCYGRLEEKGC
NIRTQSYKYRFAWGVVTCEPGPFCHVNLCACDRKLVYCLKRNLRSYNPQYQYFPNILCS
⁹⁰FAWGVVTC⁷⁷ (86)
group 10 secretory phospholipase A2 precursor [Homo sapiens]
group 10 secretory phospholipase A2 precursor [Homo sapiens]
>gi|4505845|ref|NP_003552.1|phospholipase A2, group X precursor [Homo sapiens]
MGPLPVCLPIMLLLLLPSLLLLLLLPGPGSGEASRILRVHRRGILELAGTVGCVGPRTPIAYMKY
GCFCGLGGHGQPRDAIDWCCHGHDCCYTRAEEAGCSPKTERYSWQCVNQSVLCGPAENKCQ
ELLCKCDQEIANCLAQTEYNLKYLFYPQFLCEPDSPKCD
⁵⁰TVGCV⁵⁴ (.161)
¹⁰⁷YSWQC¹¹¹ (0.29)
¹⁴⁹YLFYP¹⁵³ (0.111)
group XIIA secretory phospholipase A2 precursor [Homo sapiens]
group XIIA secretory phospholipase A2 precursor [Homo sapiens]
>gi|21361944|ref|NP_110448.2|group XIIA secretory phospholipase A2 precursor [Homo sapiens]
MALLSRPALTLLLLLMAAVVRCQEQAQTTDWRATLKTIRNGVHKIDTYLNAALDLLGGEDGL
CQYKCSDGSKPFPRYGYKPSPPNGCGSPLFGVHLNIGIPSLTKCCNQHDRCYETCGKSICNDCD
EEFQYCLSKICRDVQKTLGLTQHVQACETTVELLFDSVIHLGCKPYLDSQRAACRCHYEEKTDL
⁹LTLLLLLMAAVV²⁰ 99
¹²⁸FQYCL¹³² (0.237)
PAF acetylhydrolases
phospholipase A2, group VII precursor [Homo sapiens]
phospholipase A2, group VII precursor [Homo sapiens]
>gi|270133071|ref|NP_001161829.1|platelet-activating factor acetylhydrolase precursor
[Homo sapiens]
MVPPKLHVLFCLCGCLAVVYPFDWQYINPVAHMKSSAWVNKIQVLMAAASFGQTKIPRGNG
PYSVGCTDLMFDHTNKGTFLRLYYPSQDNDRLDTLWIPNKEYFWGLSKFLGTHWLMGNILRL
LFGSMTTPANWNSPLRPGEKYPLVVFSHGLGAFRTLYSAIGIDLASHGFIVAAVEHRDRSASAT
YYFKDQSAAEIGDKSWLYLRTLKQEEETHIRNEQVRQRAKECSQALSLILDIDHGKPVKNALD
LKFDMEQLKDSIDREKIAVIGHSFGGATVIQTLSEDQRFRCGIALDAWMFPLGDEVYSRIPQPL
FFINSEYFQYPANIIKMKKCYSPDKERKMITIRGSVHQNFADFTFATGKIIGHMLKLKGDIDSNV
AIDLSNKASLAFLQKHLGLHKDFDQWDCLIEGDDENLIPGTNINTTNQHIMLQNSSGIEKYN

| | |
|---|---|
| ⁸VLFCLCGCLAVV¹⁹ (83) | ²³³LSLIL²³⁷ (10) |
| ⁴⁴VLMAAA⁴⁹ (15) | ²⁶⁷IAVIG²⁷¹ (20.5) |
| ¹⁰³YFWGL¹⁰⁷ (15.8) | ³¹⁴LFFIN³¹⁸ (17) |
| ¹⁴⁶LVVFS¹⁵⁰ (48) | ³⁵⁷FTFAT³⁶¹ (11.4 |
| ¹⁵⁹LYSAIGI¹⁵⁵ (9.8) | |
| ¹⁷²FIVAAV¹⁷⁷ (87) | |
| ¹⁸⁶ATYYF¹⁹⁰ (18.6) | |
| ²⁰²SWLYL²⁰⁶ (8.7) | |

Myeloperoxidase
>sp|P05164|PERM_HUMAN Myeloperoxidase OS = Homo sapiens GN = MPO PE = 1 SV = 1
MGVPFFSSLRCMVDLGPCWAGGLTAEMKLLLALAGLLAILATPQPSEGAAPAVLGEVDTSLVLSSMEEAKQLVDK
AYKERRESIKQRLRSGSASPMELLSYFKQPVAATRTAVRAADYLHVALDLLERKLRSLWRRPFNVTDVLTPAQLN
VLSKSSGCAYQDVGVTCPEQDKYRTITGMCNNRRSPTLGASNRAFVRWLPAEYEDGFSLPYGWTPGVKRNGFPVA
LARAVSNEIVRFPTDQLTPDQERSLMFMQWGQLLDHDLDFTPEPAARASFVTGVNCETSCVQQPPCFPLKIPPND
PRIKNQADCIPFFRSCPACPGSNITIRNQINALTSFVDASMVYGSEEPLARNLRNMSNQLGLLAVNQRFQDNGRA
LLPFDNLHDDPCLLTNRSARIPCFLAGDTRSSEMPELTSMHTLLLREHNRLATELKSLNPRWDGERLYQEARKIV
GAMVQIITYRDYLPLVLGPTAMRKYLPTYRSYNDSVDPRIANVFTNAFRY
GHTLIQPFMFRLDNRYQPMEPNPRVPLSRVFFASWRVVLEGGIDPILRGLMATPARLNRQNQIAVDEIRERLFEQ
VMRIGLDLPALNMQRSRDHGLPGYNAWRRFCGLPQPETVGQLGTVLRNLKLARKLMEQYGTPNNIDIWMGGVSEP
LKRKGRVGPLLACIIGTQFRKLRDGDRFWWENEGVFSMQQRQALAQISLPRIICDNTGITTVSKNNIFMSNSYPR
DFVNCSTLPALNLASWREAS
²⁹LLLALAGLLAILA⁴¹
⁹⁸LLSYF¹⁰²
²⁴⁹SLMFMQWG²⁵⁶
²⁷⁵FVTGV²⁷⁹
³³³LTSFV³³⁷
³⁶⁰LGLLAV³⁶⁵
⁴⁴⁹IVGAMVQIITY⁴⁵⁹
⁴⁹³VFTNAF⁴⁹⁸
⁵³⁰VFFASWRVVLEGGI⁵⁴³
Acid Sphingomyelinase
>gi|179095|gb|AAA58377.1|acid sphingomyelinase [Homo sapiens]
MPRYGASLRQSCPRSGREQGQDGTAGAPGLLWMGLVLALALALALALSDSRVLWAPAEAHPLSPQGHPAR
LHRIVPRLRDVFGWGNLTCPICKGLFTAINLGLKKEPNVARVGSVAIKLCNLLKIAPPAVCQSIVHLFED
DMVEVWRRSVLSPSEACGLLLGSTCGHWDIFSSWNISLPTVPKPPPKPPSPPAPGAPVSRILFLTDLHWD
HDYLEGTDPDCADPLCCRRGSGLPPASRPGAGYWGEYSKCDLPLRTLESLLSGLGPAGPFDMVYWTGDIP
AHDVWHQTRQDQLRALTTVTALVRKFLGPVPVYPAVGNHESIPVNSFPPPFIEGNHSSRWLYEAMAKAWE
PWLPAEALRTLRIGGFYALSPYPGLRLISLNMNFCSRENFWLLINSTDPAGQLQWLVGELQAAEDRGDKV
HIIGHIPPGHCLKSWSWNYYRIVARYENTLAAQFFGHTHVDEFEVFYDEETLSRPLAVAFLAPSATTYIG
LNPGYRVYQIDGNYSRSSHVVLDHETYILNLTQANIPGAIPHWQLLYRARETYGLPNTLPTAWHNLVYRM
RGDMQLFQTFWFLYHKGHPPSEPCGTPCRLATLCAQLSARADSPALCRHLMPDGSLPEAQSLWPRPLFC
³⁰LLWMGLVLALALALALAL⁴⁸ (99)
⁹⁵LFTAI⁹⁹ (13)
²⁰¹ILFLT²⁰⁵ (86)
¹⁹⁵ALTTVTALV³⁰³ (9)
³⁸⁹NFWLLI³⁹⁴ (86)
⁴⁷⁶LAVAFL⁴⁸¹ (25)
⁵⁶⁶LFQTFWFLY⁵⁷⁴(95)
Heat shock protein 70
>gi|292160|gb|AAA02807.1|heat shock protein 70 [Homo sapiens]
MSVVGIDLGFQSCYVAVARAGGIETIANEYSDRCTPACISFGPKNRSIGAAAKSQVISNAKNTVQGFKRF
HGRAFSDPFVEAEKSNLAYDIVQWPTGLTGIKVTYMEEERNFTTEQVTAMLLSKLKETAESVLKKPVVDC
VVSVPCFYTDAERRSVMDATQIAGLNCLRLMNETTAVALAYGIYKQDLPRLEEKPRNVVFVDMGHSAYQV
SVCAFNRGKLKVLATAFDTTLGGRKFDEVLVNHFCEEFGKKYKLDIKSKIRALLRLSQECEKLKKLMSAN
ASDLPLSIECFMNDVDVSGTMNRGKFLEMCNDLLARVEPPLRSVLEQTKLKKEDIYAVEIVGGATRIPAV
KEKISKFFGKELSTTLNADEAVTRGCALQCAILSPAFKVREFSITDVVPYPISLRWNSPAEEGSSDCEVF
SKNHAAPFSKVLTFYRKEPFTLEAYYSSPQDLPYPDFAIAQFSVQKVTFQSDGSSSKVKVKVRVNVHGIF
SVSSASLVEV
HKSEENEEPMETDQNAKEEEKMQVDQEEPHVEEQQQQTPAENKAESEEMETSQAGSKDKK
MDQPPQCQEGKSEDQYCGPANRESAIWQIDREMLNLYIENEGKMIMQDKLEKERNDAKNAVEEYVYEMRD
KLSGEYEKFVSEDDRNSFTLKLEDTENWLYEDGEDQPKQVYVDKLAELKNLGQPIKIRFQESEERPNYLK
N
¹⁴YVAVA¹⁸ (15.5)
¹¹⁷VTAMLL¹²² (36)
¹⁷⁴TTAVALAYGIY¹⁸⁴ (65)
¹⁹⁷NVVFV²⁰¹ (66)
²²²VLATAF²²⁷ (49)
⁵⁹³MLNLYI⁵⁹⁸ (5)

### REFERENCES

Atherton, E.; Sheppard, R. C., 1989, Solid Phase peptide synthesis: a practical approach. Oxford, England: IRL Press. ISBN 0199630674.
Code, Christian, Domanov, Yegor A., Jutila, Arimatti, and Kinnunen, Paavo K. J., 2008, Amyloid-Type Fiber Formation in Control of Enzyme Action: Interfacial Activation of Phospholipase A₂, Biophysical Journal, 95:215-224
Code, Christian, Domanov, Yegor A., Killian, J. Antoinette, and Kinnunen, Paavo K. J., 2009, Activation of phospholipase A₂ by temporin B: Formation of antimicrobial peptide-enzyme amyloid-type cofibrils, BBA—Biomembranes, doi:10.1016/j.bbamem.2009.03.002
Conchillo-Solé, Oscar, de Groot, Natalia S., Aviles, Francesc X., Vendrell, Josep, Daura, Xavier, and Ventura, Salvador, 2007, AGGRESCAN: a server for the prediction and evaluation of “hot spots” of aggregation in polypeptides, BMC Bioinformatics, 8:65; doi 10.1186/1471-2105-8-65
Fernandez-Escamilla A M, Rousseau F, Schymkowitz J, and Serrano L, 2004, Prediction of sequence-dependent and mutational effects on the aggrgation of peptides and proteins, Nat Biotechnol, e-pub
Linding R, Schymkowitz J, Rousseau F, diella F, and Serrano L, 2004, A comparative study of the relationship between protein structure and beta-aggregation in globular and intrinsically disordered proteins, J Mol Biol 354-353
Rousseau, Frederic, Schymkowitz, Joost, and Serrano, Luis, 2006, Protein aggregation and amyloidosis: confusion of the kinds?, Current Opinion in Structural Biology, 16:1-9
Shimizu, Takao, 2009, Lipid Mediators in Health an Disease: Enzymes and Receptors as Therapeutic Targets for the Regulation on Immunity and Inflammation, Annu. Rev. Pharmacol. Toxicol., 49:123-150
Trovato, Antonio, Seno, Flavio, and Tosatto, Silvio C. E., 2007, The PASTA server for protein aggregation prediction, Protein Engineering Design and Selection, 20(10):521-523; doi:10.1093/protein/gzm042

1. A method for preparing peptide inhibitors of a lipid-activated enzyme, the method comprising the steps of:
a) identifying aggregation-prone regions in amino acid sequence of said enzyme by the use of a suitable computer algorithm;
b) designing a peptide based on the aggregation-prone region found in step a), wherein said peptide comprises the sequence of said region or a part thereof;
c) synthesizing the peptide designed in step b); and
d) contacting the peptide obtained in step c) with said lipid-activated enzyme and measuring the activity of said enzyme, wherein said peptide is an inhibitor of said enzyme, if the activity of the enzyme is decreased in the presence of said peptide.

2. The method according to claim 1, wherein said lipid-activated enzyme is selected from the group consisting of phospholipases, myeloperoxidase, acid sphingomyelinase, heat shock protein 70 and PAF acetylhydrolase.

3. The method according to claim 2, wherein said lipid-activated enzyme is bee-venom phospholipase A2 and the peptide designed in step b) is SYFVGKMYFNLI (SEQ ID NO:2).

4. The method according to claim 2, wherein said lipid-activated enzyme is phospholipase A2 in human tears and the peptide designed in step b) is TKFLSYK (SEQ ID NO:3).

5. The method according to claim 2, wherein said lipid-activated enzyme is myeloperoxidase and the peptide designed in step b) is selected from the group consisting of:
(SEQ ID NO: 4)
LLLALAGLLAILA,
(SEQ ID NO: 5)
LLSYF,
(SEQ ID NO: 6)
SLMFMQWG,
(SEQ ID NO: 7)
FVTGV,
(SEQ ID NO: 8)
LTSFV,
(SEQ ID NO: 9)
LGLLAV,
(SEQ ID NO: 10)
IVGAMVQIITY,
(SEQ ID NO: 11)
VFTNAF,
and
(SEQ ID NO: 12)
VFFASWRVVLEGGI.

6. The method according to claim 2, wherein said lipid-activated enzyme is acid sphingomyelinase and the peptide designed in step b) is selected from the group consisting of:
(SEQ ID NO: 13)
LLWMGLVLALALALALAL,
(SEQ ID NO: 14)
LFTAI,
(SEQ ID NO: 15)
ILFLT,
(SEQ ID NO: 16)
ALTTVTALV,
(SEQ ID NO: 17)
NFWLLI,
(SEQ ID NO: 18)
LAVAFL,
and
(SEQ ID NO: 19)
LFQTFWFLY.

7. The method according to claim 2, wherein said lipid-activated enzyme is heat shock protein 70 and the peptide designed in step b) is selected from the group consisting of:
(SEQ ID NO: 20)
YVAVA,
(SEQ ID NO: 21)
VTAMLL,
(SEQ ID NO: 22)
TTAVALAYGIY,
(SEQ ID NO: 23)
NVVFV,
(SEQ ID NO: 24)
VLATAF,
and
(SEQ ID NO: 25)
MLNLYI.

8. The method according to claim 2, wherein said lipid-activated enzyme is PAF acetylhydrolase and the peptide designed in step b) is selected from the group consisting of:
(SEQ ID NO: 26)
VLFCLCGCLAVV,
(SEQ ID NO: 27)
VLMAAA,
(SEQ ID NO: 28)
YFWGL,
(SEQ ID NO: 29)
LVVFS,
(SEQ ID NO: 30)
LYSAIGI,
(SEQ ID NO: 31)
FIVAAV,
(SEQ ID NO: 32)
ATYYF,
(SEQ ID NO: 33)
SWLYL,
(SEQ ID NO: 34)
SWLYL,
(SEQ ID NO: 35)
LSLIL,
(SEQ ID NO: 36)
IAVIG,
(SEQ ID NO: 37)
LFFIN,
and
(SEQ ID NO: 38)
FTFAT.

9. The method according to claim 2, wherein said lipid-activated enzyme is human phospholipase A2 and the peptide designed in step b) is selected from the group consisting of:
(SEQ ID NO: 39)
AAICF,
(SEQ ID NO: 40)
AALSYGFY,
(SEQ ID NO: 41)
VAFCLK,
(SEQ ID NO: 42)
VQFGVMI,
(SEQ ID NO: 43)
YLFSVS,
(SEQ ID NO: 44)
AILSFVGY,
(SEQ ID NO: 45)
VLCLM,
(SEQ ID NO: 46)
FLNVY,
(SEQ ID NO: 47)
LFGMLGFLGVAL,
(SEQ ID NO: 48)
LGYLSFLA,
(SEQ ID NO: 49)
IVGVAFFNVL,
(SEQ ID NO: 50)
ACVAWYWW,
(SEQ ID NO: 51)
FTVVVL,
(SEQ ID NO: 52)
ATFTV
(SEQ ID NO: 53)
VVAIL,
(SEQ ID NO: 54)
WYMSTLY,
(SEQ ID NO: 55)
IFGMLI,
(SEQ ID NO: 56)
LFTCL,
(SEQ ID NO: 57)
FFMGTV,
(SEQ ID NO: 58)
FLMGVWGSAFSILF,
(SEQ ID NO: 59)
MIMALV,
(SEQ ID NO: 60)
LIISF,
(SEQ ID NO: 61)
TIIHFVLANI
(SEQ ID NO: 62)
FAWGVVTC,
(SEQ ID NO: 63)
TVGVC,
(SEQ ID NO: 64)
YSWQC,
(SEQ ID NO: 65)
YLFYP,
(SEQ ID NO: 66)
LTLLLLLMAAVV
and
(SEQ ID NO: 67)
FQYCL.

10. A peptide comprising an amino acid sequence selected from the group consisting of SEQ ID NOS:1-68.

11. The peptide according to claim 10 comprising amino acid sequence KMYFNLI (SEQ ID NO:1).

12. The peptide according to claim 11 consisting of amino acid sequence KMYFNLI (SEQ ID NO:1).

13. The peptide according to claim 10 comprising amino acid sequence AALSYGFYG (SEQ ID NO:68).

14. The peptide according to claim 13 consisting of amino acid sequence AALSYGFYG (SEQ ID NO:68).

15. The peptide according to claim 10, wherein said peptide is chemically associated or bonded to the transporter peptide.

16. The peptide according to claim 11, wherein said peptide is chemically associated or bonded to the transporter peptide.

17. The peptide according to claim 12, wherein said peptide is chemically associated or bonded to the transporter peptide.

18. The peptide according to claim 13, wherein said peptide is chemically associated or bonded to the transporter peptide.

19. The peptide according to claim 14, wherein said peptide is chemically associated or bonded to the transporter peptide.
